SEQUENCE LISTING (18 sequences)

SEQ ID NO: 1 - DNA, 21 bp, Artificial Sequence
  oligonucleotide
ttgctcgacgaattactgctc

SEQ ID NO: 2 - DNA, 27 bp, Homo sapiens
tggtaattgctcgacgaattactgctc

SEQ ID NO: 3 - protein, 9 aa, Homo sapiens
TINELLNDE

SEQ ID NO: 4 - DNA, 20 bp, Artificial Sequence
  oligonucleotide
aattgctcgacgaattactg

SEQ ID NO: 5 - DNA, 20 bp, Artificial Sequence
  oligonucleotide
tggagctgctgaacgacgag

SEQ ID NO: 6 - DNA, 27 bp, Artificial Sequence
  oligonucleotide
tggtaattggagctgctgaacgacgag

SEQ ID NO: 7 - DNA, 22 bp, Artificial Sequence
  oligonucleotide
gtatggccaaactggcactgga

SEQ ID NO: 8 - DNA, 40 bp, Homo sapiens
gtatggccaaactggcactggaaaaacttttacaatggaa

SEQ ID NO: 9 - protein, 13 aa, Homo sapiens
VWPNWHWKNFYNG

SEQ ID NO: 10 - DNA, 20 bp, Artificial Sequence
  oligonucleotide
cactggaaaaacttttacaa

SEQ ID NO: 11 - DNA, 22 bp, Artificial Sequence
  oligonucleotide
gtttggcctaattggcattgga

SEQ ID NO: 12 - DNA, 40 bp, Artificial Sequence
  oligonucleotide
gtttggcctaattggcattggaagaccttcaccaatggaa

SEQ ID NO: 13 - DNA, 22 bp, Artificial Sequence
  oligonucleotide
cacaccgttgaaaagggaggca

SEQ ID NO: 14 - DNA, 35 bp, Homo sapiens
cacaccgttgaaaagggaggcaagcacaagactgg

SEQ ID NO: 15 - protein, 11 aa, Homo sapiens
HTVEKGGKHKT

SEQ ID NO: 16 - DNA, 20 bp, Artificial Sequence
  oligonucleotide
aagggaggcaagcacaagac

SEQ ID NO: 17 - DNA, 33 bp, Artificial Sequence
  oligonucleotide
catactgtggagaaaggcggcaagcacaagacc

SEQ ID NO: 18 - DNA, 33 bp, Artificial Sequence; identical to SEQ ID NO: 17
  oligonucleotide

(19) United States
(12) Patent Application Publication
(10) Pub. No.: US 2022/0251566 A1
(43) Pub. Date: Aug. 11, 2022

(54) CELLS ENGINEERED FOR OLIGONUCLEOTIDE DELIVERY, AND METHODS FOR MAKING AND USING THEREOF

(71) Applicant: The Research Foundation for The State University of New York, Albany, NY (US)

(72) Inventors: Sergey V. DORONIN, Stony Brook, NY (US); Irina A. POTAPOVA, Stony Brook, NY (US); Ira S. COHEN, Stony Brook, NY (US)

(73) Assignee: The Research Foundation for The State University of New York, Albany, NY (US)

(21) Appl. No.: 17/622,355

(22) PCT Filed: Jun. 25, 2020
(86) PCT No.: PCT/US2020/039657

§ 371 Date: Dec. 23, 2021

Related U.S. Application Data
(60) Provisional application No. 62/866,940, filed on Jun. 26, 2019.

(51) Int. Cl. C12N 15/113; C12N 15/90; C12N 15/11; C12N 9/22; A61K 35/28

(52) U.S. Cl. CPC C12N 15/1135; C12N 15/907; C12N 15/11; C12N 9/22; C12N 2800/80; C12N 2320/32; C12N 2310/14; C12N 2310/20; A61K 35/28

(57) ABSTRACT

The present disclosure is directed to genetically modified carrier/donor cells that are engineered to be resistant to its oligonucleotide payload, and methods of delivering an oligonucleotide into a target cell. The disclosure is also directed to methods of treating cancer using the engineered carrier cells of the disclosure. An aspect of this disclosure is directed to engineering carrier cells to be resistant to the detrimental effects of an oligonucleotide.

### CROSS REFERENCE TO RELATED APPLICATION

This application claims the benefit of priority from U.S. Provisional Application No. 62/866,940, filed Jun. 26, 2019, the entire contents of which are incorporated herein by reference.

### INCORPORATION BY REFERENCE OF SEQUENCE LISTING

The Sequence Listing in an ASCII text file, named as 38479 (050-9081).PCT.seqlist_ST25.txt of 4 KB, created on Jun. 22, 2020, and submitted to the United States Patent and Trademark Office via EFS-Web, is incorporated herein by reference.

### BACKGROUND

Oligonucleotide-based therapies have been limited in application, partly due to lack of appropriate methods for delivery.

Oligonucleotides, either single or double stranded, can be passed through gap junctions formed by connexin proteins, as demonstrated by a single electrode delivery of fluorescent-tagged oligonucleotides to a carrier cell (aka. a “donor cell”) and determining their transfer to the target cell via gap junction mediated communication. See, e.g., U.S. Pat. Nos. 8,188,062; 7,842,673; 7,794,702, all of which are incorporated herein in their entirety. Oligonucleotides can also be passed from one cell to another by the exosome pathway, where the carrier/donor cell can release the oligonucleotide cargo for the target/recipient cell to take in by endocytosis.

Oligonucletides, such as small interfering RNAs (siRNAs) and antisense oligonucleotides (ASO), have therapeutic potential. Knowledge of the human genome allows the development of drugs based on siRNAs or ASOs and the use of these drugs for the regulation of the activity of any gene. However, practical application of siRNAs or ASOs is limited by two factors. First is the lack of a reliable delivery system and, second is the likelihood of toxicity from systemic delivery. Thus, the set of genes available for regulation by siRNA or ASO is limited to a small number of known targets that exhibit mild or are absent of systemic toxicity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C. Engineering a cell to be resistant to a specific PLK1 siRNA. (A) Sequences before gene editing and the strategy. Sequence of the PLK1 siRNA, against which the cell will be rendered resistant, is shown by SEQ ID NO: 1. The wild type PLK1 gene sequence (the target sequence=targeted by the siRNA designated as SEQ ID NO: 1) is shown by SEQ ID NO: 2. The protein translation of the target sequence is shown by SEQ ID NO: 3. The CRISPR guide RNA that targets the region for gene editing is shown by SEQ ID NO: 4. Finally, the single stranded donor DNA (ssODN) that is used for homologous recombination (that introduces the silent mutations) is shown by SEQ ID NO: 5. (B) Sequences after gene editing. The sequence of the modified PLK1 gene in the carrier cell is shown by SEQ ID NO: 6. The alignment of the final PLK1 gene sequence demonstrates that the PLK1 siRNA shown by SEQ ID NO: 1 can no longer target the final/edited sequence, while the protein sequence remains unaltered. (C) Human mesenchymal stem cells (hMSCs) were edited in the PLK1 gene as described in (A) and (B), resulting in engineered/mutant hMSCs (mt-hMSCs). Both wild type hMSCs (wt-hMSCs) and mt-HMCs were loaded with PLK1-11 siRNA (SEQ ID NO: 1) or a different PLK1 siRNA (labeled PLK1-2), and percent of cell death was measured. The engineered cells were resistant to the toxic/cell killing effects of PLK1-11 siRNA (the siRNA they were engineered against), but they were not resistant to PLK1-2 siRNA.

FIGS. 2A-2B. Engineering a cell to be resistant to a specific KIF11 siRNA. (A) Sequences before gene editing and the strategy. Sequence of the KIF11 siRNA, against which the cell will be rendered resistant, is shown by SEQ ID NO: 7. The wild type KIF11 gene sequence (the target sequence=targeted by the siRNA designated as SEQ ID NO: 7) is shown by SEQ ID NO: 8. The protein translation of the target sequence is shown by SEQ ID NO: 9. The CRISPR guide RNA that targets the region for gene editing is shown by SEQ ID NO: 10. Finally, the single stranded donor DNA (ssODN) that is used for homologous recombination (that introduces the silent mutations) is shown by SEQ ID NO: 11. (B) Sequences after gene editing. The sequence of the modified KIF11 gene in the carrier cell is shown by SEQ ID NO: 12. The alignment of the final KIF11 gene sequence demonstrates that the PLK1 KIF11 shown by SEQ ID NO: 7 can no longer target the final/edited sequence, while the protein sequence remains unaltered.

FIGS. 3A-3B. Engineering a cell to be resistant to a specific CYCS siRNA. (A) Sequences before gene editing and the strategy. Sequence of the CYCS siRNA, against which the cell will be rendered resistant, is shown by SEQ ID NO: 13. The wild type CYCS gene sequence (the target sequence=targeted by the siRNA designated as SEQ ID NO: 13) is shown by SEQ ID NO: 14. The protein translation of the target sequence is shown by SEQ ID NO: 15. The CRISPR guide RNA that targets the region for gene editing is shown by SEQ ID NO: 16. Finally, the single stranded donor DNA (ssODN) that is used for homologous recombination (that introduces the silent mutations) is shown by SEQ ID NO: 17. (B) Sequences after gene editing. The sequence of the modified CYCS gene in the carrier cell is shown by SEQ ID NO: 18. The alignment of the final CYCS gene sequence demonstrates that the PLK1 CYCS shown by SEQ ID NO: 13 can no longer target the final/edited sequence, while the protein sequence remains unaltered.

### DETAILED DESCRIPTION

### Definitions

The term “connexin” as used herein refers to a large family of trans-membrane proteins that allow intercellular communication and the transfer of ions and small signaling molecules and assemble to form gap junctions. Connexins are four-pass transmembrane proteins with both C and N cytoplasmic termini, a cytoplasmic loop (CL) and two extracellular loops, (EL-1) and (EL-2). Connexins are assembled in groups of six to form hemichannels, or connexons, and two hemichannels, one on each cell, then combine to form a gap junction between the two cells. The connexin gene family is diverse, with twenty-one identified members in the sequenced human genome, and twenty in the mouse (nineteen of which are orthologous pairs). They usually weigh between 26 and 60 kiloDaltons (kDa), and have an average length of 380 amino acids. The various connexins have been observed to combine into both homomeric gap junctions (both connexins the same) and heteromeric gap junctions (two different connexins), each of which may exhibit different functional properties including pore conductance, size selectivity, charge selectivity, voltage gating, and chemical gating. The term Connexin is abbreviated as Cx and the gene encoding for it CX. Connexins are commonly named according to their molecular weights, e.g. Cx26 is the connexin protein of 26 kDa, using the weight of the human protein for the numbering of orthologous proteins in other species.

The term “gap junction” as used herein refers to a specialized intercellular connection between a multitude of animal cell-types. They directly connect the cytoplasm of two cells, which allows various molecules, ions and electrical impulses to directly pass through a regulated gate between cells.

The term “syncytial” refers to a tissue that is made up of cells interconnected by specialized membrane with gap junctions, which are synchronized electrically in an action potential. Syncytial cells include a cardiac myocyte, a smooth muscle cell, an epithelial cell, a connective tissue cell, or a syncytial cancer cell.

The term “delivering” or “delivered” as used herein refers to introducing a molecule into an inside of a cell membrane.

The phrase “donor cell” or “carrier cell” as used herein refers to a cell that has been loaded with a molecule to be delivered to a different cell called a target cell.

The term “DNA” or “deoxyribonucleic acid” as used herein refers to a molecule made up of certain nucleic acid bases. DNA can carry most of the genetic instructions used in the development, functioning and reproduction of all known living organisms and many viruses. DNA is a nucleic acid; alongside proteins and carbohydrates, nucleic acids compose the three major macromolecules essential for all known forms of life. Most DNA molecules consist of two biopolymer strands coiled around each other to form a double helix. The two DNA strands are known as polynucleotides since they are composed of simpler units called nucleic acid bases, or more simply, nucleotides. Each nucleotide is composed of a nitrogen-containing nucleobase-either cytosine (C), guanine (G), adenine (A), or thymine (T)—as well as a monosaccharide sugar called deoxyribose and a phosphate group. The nucleotides are joined to one another in a chain by covalent bonds between the sugar of one nucleotide and the phosphate of the next, resulting in an alternating sugar-phosphate backbone.

According to base pairing rules (A with T, and C with G), hydrogen bonds bind the nitrogenous bases of the two separate polynucleotide strands to make double-stranded DNA.

The term “RNA” or “ribonucleic acid” as used herein refers to a polymeric molecule, often implicated in various biological roles in coding, decoding, regulation, and expression of genes. RNA, like DNA, is a nucleic acid. RNA is a linear molecule composed of four types of smaller molecules called ribonucleotide bases: adenine (A), cytosine (C), guanine (G), and, in place of thymine (T) found in DNA, uracil (U).

The term “gene” refers to the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

The phrase “target cell” as used herein refers to a cell selectively affected by a particular agent, such as a donor cell or content carried by the donor cell.

The terms “individual,” “subject,” “host,” and “patient,” are used interchangeably herein and refer to any mammalian subject for whom diagnosis, treatment, or therapy is desired, particularly humans.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### The CRISPR-Cas9 System

The CRISPR-Cas9 system includes a short noncoding gRNA that has two molecular components: a target-specific CRISPR RNA (crRNA) and an auxiliary trans-activating crRNA (tracrRNA). The gRNA unit guides the Cas9 nuclease to a specific genomic locus, and the Cas9 protein induces a double-stranded break at the specific genomic target sequence. Following CRISPR-Cas9-induced DNA cleavage, the double-stranded break can be repaired by the cellular repair machinery using either nonhomologous end joining (NHEJ) or a homology-directed repair mechanism (HDR).

Homology-directed repair (HDR) allows mutagenesis using a DNA repair template, such as single-stranded oligo DNA nucleotide (ssODN). Nonhomologous end joining (NHEJ) allows either deletion of a portion of the targeted gene or insertion of an arbitrary DNA sequence when conducted in the presence of double stranded blunt ended DNA.

### Oligonucleotides

The term “oligonucleotide” as used herein refers to a DNA or RNA molecule. Oligonucleotides readily bind, in a sequence-specific manner, to their respective complementary oligonucleotides, DNA, or RNA to form duplexes.

In some embodiments, the oligonucleotide of the present disclosure is between 12-40 nucleotides in length. In some embodiments, the oligonucleotide of the present disclosure is between 16-24 nucleotides in length. In some embodiments, the oligonucleotide of the present disclosure is between 20 and 30 nucleotides in length.

The term “inhibitory oligonucleotide” refers to any oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include isolated or synthetic nucleic acids such as shRNA or DNA, siRNA or DNA, antisense RNA or DNA, chimeric Antisense DNA or RNA, miRNA and miRNA mimics, among others.

The term “synthetic nucleic acid” refers to that the nucleic acid does not have a chemical structure or sequence of a naturally occurring nucleic acid. Synthetic nucleotides include an engineered nucleic acid such as a DNA or RNA molecule. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA. Consequently, it will be understood that the term “synthetic miRNA” refers to a “synthetic nucleic acid” that functions in a cell or under physiological conditions as a naturally occurring miRNA.

In some embodiments, the oligonucleotide is an RNA molecule that can traverse a gap junction or be transcribed into a peptide that can traverse a gap junction. In some embodiments, the oligonucleotide is a DNA molecule. In some embodiments, the oligonucleotide is an antisense oligonucleotide or a cDNA that produces an antisense oligonucleotide that can traverse a gap junction. In some embodiments, the oligonucleotide is a siRNA oligonucleotide or a cDNA that produces a siRNA oligonucleotide that can traverse a gap junction. In some embodiments, the oligonucleotide is a DNA or RNA that produces a peptide that can traverse the gap junction.

As used herein, the phrase “isolated nucleotide” refers to a nucleotide that is altered or removed from the natural state through human intervention.

The term “antisense” as used herein refers to a sequence of nucleotides complementary to and therefore capable of binding to a coding sequence, which may be either that of the strand of a DNA double helix that undergoes transcription, or that of a messenger RNA molecule. Antisense DNA is the non-coding strand complementary to the coding strand in double-stranded DNA. The antisense strand serves as the template for messenger RNA (mRNA) synthesis.

The terms “nucleic acid” and “nucleic acid molecule” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides.

The term “hybridization” refers to hydrogen bonding, which may be Watson-Crick, Hoogsteen or reversed Hoogsteen hydrogen bonding, between complementary nucleoside or nucleotide bases in one or more nucleotides.

The term “mRNA” or “messenger RNA” as used herein refers to the template for protein synthesis via translation and is a large family of RNA molecules that convey genetic information from DNA to the ribosome, where they specify the amino acid sequence of the protein products of gene expression.

The term small interfering RNA (siRNA), sometimes known as short interfering RNA or silencing RNA, is a class of double-stranded RNA molecules, 16-24 base pairs in length. Various siRNA plays many roles, but it is most notable in the RNA interference (RNAi) pathway, where it interferes with the expression of specific genes with complementary nucleotide sequences. An siRNA functions by causing mRNA to be broken down after transcription, resulting in no translation into a protein. An siRNA that prevents translation to a particular protein is indicated by the protein name coupled with the term siRNA. Thus an siRNA that interferes with the translation to the important kinase Akt is indicated by the expression “Akt siRNA.” Typically, an siRNA in various embodiments is a double-stranded nucleic acid molecule comprising two nucleotide strands, each strand having about 19 to about 28 nucleotides (i.e. about 19, 20, 21, 22, 23, 24, 25, 26, 27, or 28 nucleotides).

The term micro RNA (abbreviated miRNA) is a small non-coding RNA molecule (containing about 22 nucleotides) that functions in RNA silencing and post-transcriptional regulation of gene expression. The miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA. Under a standard nomenclature system, names are assigned to experimentally confirmed miRNAs. The prefix “miR” is followed by a dash and a number, the latter often indicating order of naming “MIR” refers to the gene that encodes a corresponding miRNA. Different miRNAs with nearly identical sequences except for one or two nucleotides are annotated with an additional lower case letter.

The term “miRNA mimics,” as used herein, refers to small, double-stranded RNA molecules, such as siRNA, designed to mimic endogenous mature miRNA molecules when introduced into cells.

### Carrier Cells

As used herein, “a carrier cell” is a cell capable of forming a gap junction with a target cell, or a cell capable of transferring cargo (e.g., oligonucleotides to a nearby target cell) via the exosome pathway. In some embodiments, the carrier cell is a mammalian cell. Every mammalian cell type is capable of transferring cargo (e.g., oligonucleotides to a nearby target cell) via the exosome pathway. Therefore, in some embodiments, the carrier cell can be any mammalian cell. In some embodiments, the cell is a stem/precursor cell. In some embodiments, the stem cell is a mesenchymal stem cell. In some embodiments, the carrier cell is a human cell. In some embodiments, the stem cell is a human mesenchymal stem cell.

The term “human mesenchymal stem cell” (hMSC), as used herein, refers to a human multipotent stromal cell that can differentiate into a variety of cell types, including: human osteoblasts (bone cells), human chondrocytes (cartilage cells), human myocytes (muscle cells) and human adipocytes (fat cells). In some embodiments, administered hMSCs home to cancerous sites, or sites with inflammation.

In some embodiments, a carrier cell is a cell containing or engineered to contain connexin proteins. In some embodiments, a gap junction channel is composed of one or more of connexin 43, connexin 40, connexin 45, connexin 32 and connexin 37.

In some embodiments, a carrier cell delivers its payload (oligonucleotides it carries) by forming gap junctions with a target cell or a group of target cells. In some embodiments, the target cell is within a syncytial (or interconnected) tissue. In some embodiments, the syncytial tissue is selected from cardiac myocyte, a smooth muscle cell, an epithelial cell, a connective tissue cell, or a syncytial cancer cell. In some embodiments, oligonucleotides delivered to one cell in the syncytial tissue can diffuse/move between the connected cells.

In some embodiments, a carrier cell delivers its payload/cargo (oligonucleotides it carries) through the exosome/endosome pathway, wherein the carrier cell releases the payload (i.e., the carried oligonucleotide) through exocytosis in an exosome and the target cell/target cells take up the payload through endocytosis.

### GENERAL DESCRIPTION

### Methods for Engineering Carrier (Donor) Cells

Cell-based delivery of oligonucleotides eliminates systemic toxic effects of inhibitory oligonucleotides (e.g., siRNA, ASO, miRNA, etc.). However, cell-based delivery does not permit delivery of any oligonucleotide, because oligonucleotides which are toxic for target cells are likely to be toxic for the carrier cell as well. In some instances, oligonucleotides which are toxic for target cells may not kill the carrier cell, but may cause it to act in undesirable ways (e.g., the carrier cell may not locate to the target site, or may release toxic substances, as a result of the oliugonucleotide cargo). As such, a cell-based method is suitable for short-term delivery of siRNA. This lifetime is limited by: 1) the half life (stability) of siRNA loaded into the carrier cell; and 2) the ability of the carrier cell to survive a payload.

An aspect of this disclosure is directed to engineering carrier cells to be resistant to the detrimental effects of an oligonucleotide. The methods of the instant disclosure can be used to genetically engineer any carrier cell to be resistant to any given oligonucleotide. Therefore, the engineered carrier cells of the disclosure can carry the oligonucleotide they are resistant to without being affected by the oligonucleotide. The term “engineered” when used in relation to cells herein refers to cells that have been engineered by man to result in the recited phenotype (e.g., resistant to an oligonucleotide), or to express a recited nucleic acid molecule or polypeptide. The term “engineered cells” is not intended to encompass naturally occurring cells, but is, instead, intended to encompass, for example, cells that comprise a recombinant nucleic acid molecule, or cells that have otherwise been altered artificially (e.g. by genetic modification—as defined below), for example so that they express a oligonucleotide/polypeptide that they would not otherwise express.

In some embodiments, the method comprises introducing a mutation in a carrier cell in the region of the genome of the cell targeted by a specific inhibitory oligonucleotide. In some embodiments, the introduced mutation prevents binding/hybridization of the inhibitory oligonucleotide to its target sequence in the carrier cell.

In some embodiments, the oligonucleotide targets a coding region of a gene, and the mutation in the genome of the cell comprises a silent mutation in the targeted coding region of the gene of the carrier cell. In some embodiments, the oligonucleotide targets a noncoding region of the genome, and the mutation in the genome of the cell comprises a mutation in the targeted noncoding region of the gene of the carrier cell. In some embodiment, the mutation comprises a deletion in the region of the genome of the carrier cell targeted by the oligonucleotide, thereby preventing hybridization or targeting of the oligonucleotide in the carrier cell.

In some embodiments, the mutation is introduced by genome editing techniques. In some embodiments, the genome editing method is selected from the group consisting of CRISPR/Cas system, Cre/Lox system, TALEN system, ZFNs system and homologous recombination. In some embodiments, the CRISPR-mediated genome editing comprises introducing into the cell a first nucleic acid encoding a Cas9 nuclease, a second nucleic acid comprising a guide RNA (gRNA), wherein the gRNA is specific to the region of the genome of the cell targeted by the oligonucleotide, and a third nucleic acid that will act as a template for homologous recombination, wherein the third nucleotide comprises the mutation.

### Engineered Carrier Cells

Another aspect of the disclosure is directed to a carrier cell engineered to be resistant to an oligonucleotide comprising a mutation in the region of the genome of the carrier cell targeted by the oligonucleotide.

In some embodiments, the oligonucleotide targets a coding region of a gene, and the mutation in the genome of the cell comprises a silent mutation in the targeted coding region of the gene.

In some embodiments, the oligonucleotide targets a noncoding region of the genome, and the mutation in the genome of the cell comprises a mutation in the targeted noncoding region of the gene.

In some embodiments, the mutation comprises a deletion in the region of the genome of the carrier cell targeted by the oligonucleotide.

In some embodiments, the carrier cell is any mammalian cell. In some embodiments, the carrier cell is a mesenchymal stem cell. In some embodiments, the carrier cell is a human cell. In some embodiments, the carrier cell is a human mesenchymal stem cell. In some embodiments, the carrier cell is an immortalized human mesenchymal stem cell. In some embodiments, the immortalized human mesenchymal stem cell is the ATCC cell line designated as SCRC-4000.

### Methods of Delivery and Treatment

Another aspect of the disclosure is directed to a method of delivering at least one oligonucleotide to a subject comprising administering to the subject a carrier cell loaded with the at least one oligonucleotide, wherein the carrier cell is engineered to be resistant to the at least one oligonucleotide.

In some embodiments, a method of delivering an oligonucleotide into a target cell is provided, comprising introducing an oligonucleotide into a carrier cell, and contacting the target cell with the carrier cell under conditions permitting the carrier cell to form a gap junction with the target cell, whereby the oligonucleotide is delivered into the target cell from the carrier cell.

In some embodiments, a method of delivering an oligonucleotide into a syncytial target cell is provided, comprising introducing an oligonucleotide into a carrier cell, and contacting the syncytial target cell with the carrier cell under conditions permitting the carrier cell to form a gap junction with the syncytial target cell, whereby the oligonucleotide is delivered into the syncytial target cell from the carrier cell.

In some embodiments, a method of delivering an oligonucleotide into a target cell is provided, comprising introducing an oligonucleotide into a carrier cell, and contacting the target cell with the carrier cell under conditions permitting the carrier cell to form an exosome that contains the oligonucleotide, whereby the oligonucleotide is delivered into the target cell by endocytosis.

Another aspect of the disclosure is directed to a method of treating a subject suffering from cancer comprising administering to the subject a carrier cell loaded with at least one oligonucleotide designed to kill a cancer cell, wherein the carrier cell is resistant to the killing effects of the at least one oligonucleotide.

In some embodiments, the cancer is brain cancer, bladder cancer, breast cancer, cervical cancer, colon and rectal cancer, head and neck cancer, glioblastoma multiform, hepatocellular cancer, kidney (renal) cancer, leukemia, lung cancer, non-small-cell lung cancer, melanoma, mesothelioma, non-Hodgkin lymphoma, Hodgkin lymphoma, ovarian cancer, pancreatic cancer, prostate cancer, skin cancer (non-melanoma), or thyroid cancer. In some embodiments, the cancer is lung cancer, non-small cell lung cancer, mesothelioma, brain cancer, glioblastoma multiforme, skin cancer, or melanoma.

In some embodiments, the cancer cell is part of a solid tumor.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### Examples

### Example 1: Polo-Like Kinase 1 (PLK1) Gene

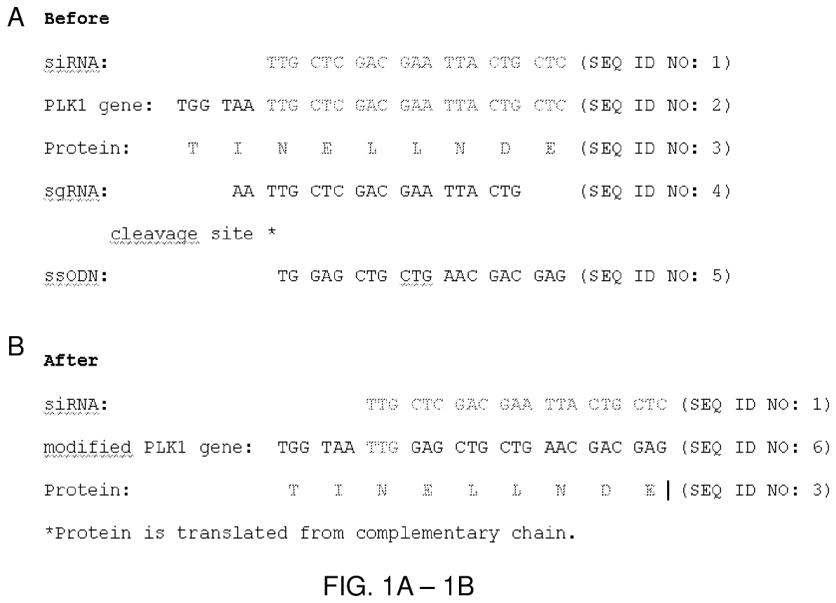

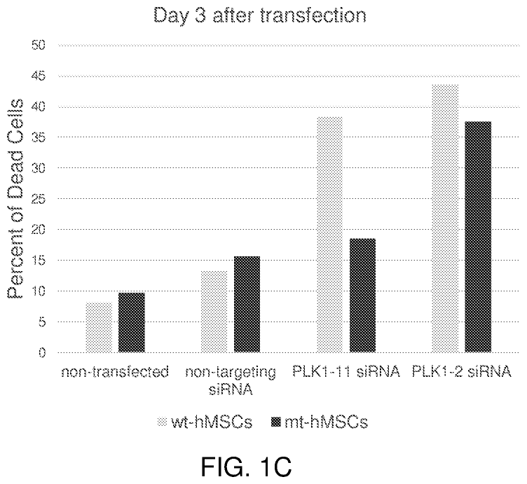

This exemplary embodiment shows how the endogenous PLK1 gene in a carrier cell can be engineered such that the gene still has the same amino acid sequence (i.e. the mutation is silent), yet the engineered mutation renders the PLK1 gene in the carrier cell resistant to the inhibitory oligonucleotide (in this example an siRNA) it carries. See FIGS. 1A-1B.

The carrier cell, after the CRISPR-mediated engineering of the endogenous PLK1 gene, becomes resistant to the load siRNA (SEQ ID NO: 1), which targets the PLK1 gene. While initially the siRNA was 100% identical to the endogenous target site (FIG. 1A), after the engineering (bases marked in red), after the modification, only the first codon is left unchanged, while the rest of the codons were all swapped with alternative codons that encode the same amino acid as the codon they have replaced (FIG. 1B). With so many mismatches, the siRNA (SEQ ID NO: 1) can no longer target the engineered PLK1 gene. The protein sequence of the PLK1 gene of the carrier cell is not altered (shown as SEQ ID NO: 3 in FIGS. 1A and 1B). As a result, the engineered cell can carry the PLK1 siRNA and deliver it to a target cell without being affected by the negative effects of the siRNA.

FIG. 1C demonstrates the protection of engineered carrier cells to a PLK1 siRNA. In this example, human mesenchymal stem cells (hMSCs) were used as carrier cells. The inventors rendered some hMSCs resistant to a specific PLK1 siRNA (shown as PLK-11 siRNA in the figure) as described above (labeled “mt-hMSCs”—“mt” stands for mutant). When loaded with PLK1-11 siRNA, there was significantly less cell death in the engineered carrier cells (mt-hMSCs) as compared to wild type counterparts loaded with the same siRNA. See FIG. 1C, third columns. However, the carrier cells were as sensitive to a different PLK1 siRNA (labeled PLK1-2) and showed similar death percentages when loaded with the different siRNA. See FIG. 1C, fourth columns.

### Example 2: Kinesin Family Member 11 (KIF11) Gene

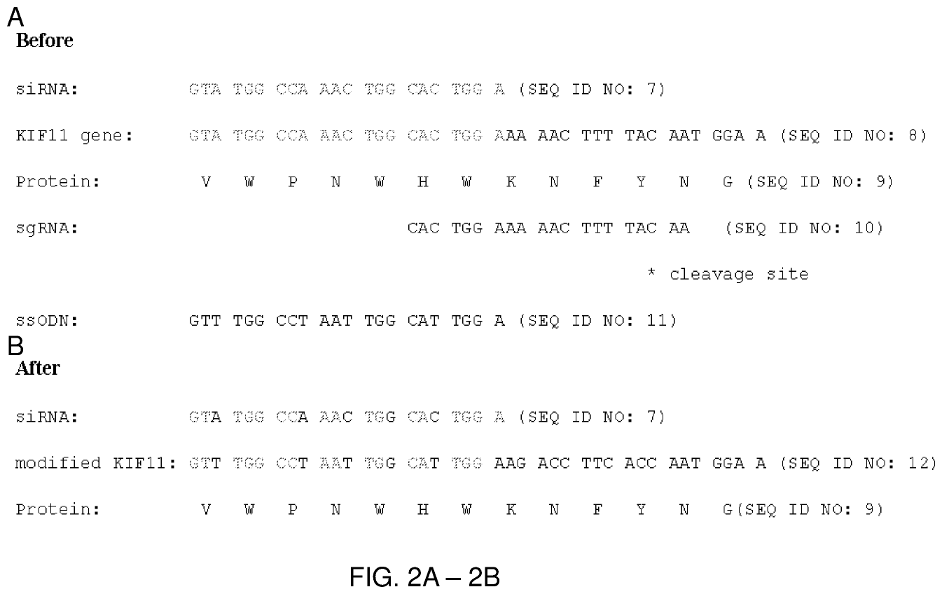

Another exemplary design directed to engineering the endogenous KIF11 gene of a carrier cell to be resistant to a specific KIF11 siRNA (SEQ ID NO: 7) is shown in FIGS. 2A-2B. FIG. 2A shows the original endogenous KIF11 sequence (SEQ ID NO: 8) before gene editing. FIG. 2B shows that, after gene editing, the KIF11 siRNA can no longer target the edited KIF11 gene (SEQ ID NO: 12) of the carrier cell. Sequences in red show the bases that are the same between the siRNA and the corresponding endogenous sequence.

### Example 3: Cytochrome C (CYCS)

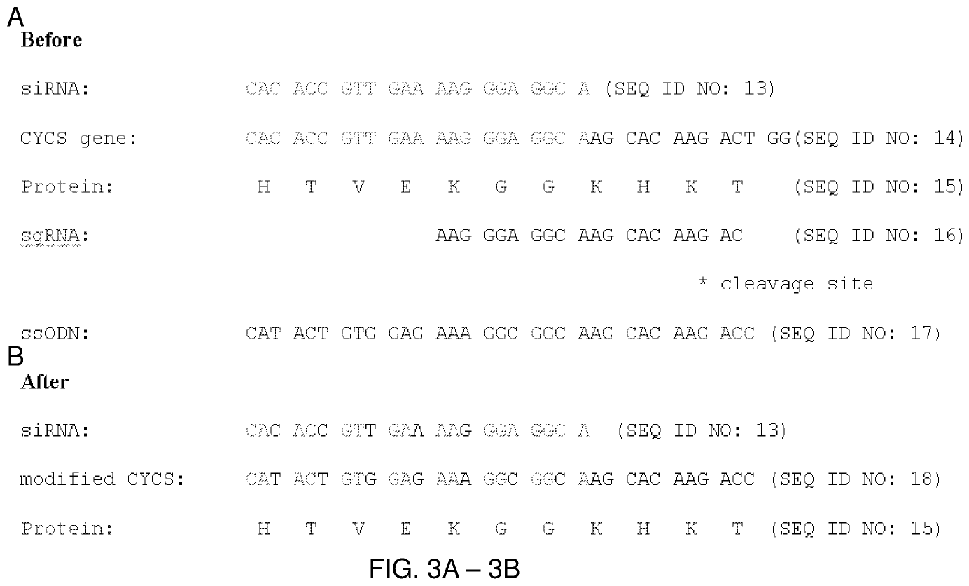

Another exemplary design directed to engineering the endogenous CYCS gene of a carrier cell to be resistant to a CYCS siRNA is shown in FIGS. 3A-3B. CYCS is an essential gene for cellular respiration, and knockdown or knockout of CYCS is akin cyanide poisoning. Therefore, normally, a carrier cell cannot deliver an siRNA targeting the CYCS, because the same siRNA would be toxic for the carrier cell.

FIG. 3A shows the original endogenous CYCS sequence before gene editing. FIG. 3B shows that, after gene editing, the CYCS siRNA can no longer target the edited CYCS gene of the carrier cell. Sequences in red show the bases that are the same between the siRNA and the corresponding endogenous sequence.

### Methods Used in the Examples

Genome Editing (Gene Knock-In)
1. Design CRISPR gRNA.
2. Transfect cells with Cas9 Protein+gRNA Reagent±recombinant DNA using the Lipofectamine CRISPRMAX Cas9 Transfection or nucleoporation.
3. Select survival cells after treatment with desired siRNA.
4. Validate gene knock-in using sequencing

Transfection Protocol Using Lipofectamine CRISPRMAX CAS9 Transfection.
1. Seed the cells the day before. Use cells at 30-70% confluence on the day of transfection when using lipid-mediated delivery (or 70-90% confluence for electroporation).
2. Prepare Tube 1: TrueCut™ Cas9 Protein v2+gRNA solution with Cas9 Plus™ Reagent in Opti-MEM™ I Medium according manufacturing recommendations.
3. Prepare Tube 2: Dilute Lipofectamine™ CRISPRMAX™ reagent in Opti-MEM™ I Medium
4. Mix Tube 1+Tube 2, incubate 15 min at RT
   Add the transfection complex to cells and incubate at 37° C.

While the invention has been shown and described with reference to certain embodiments of the present invention thereof, it will be understood by those skilled in the art that various changes in from and details may be made therein without departing from the spirit and scope of the present invention and equivalents thereof.

What is claimed is:

1. A method for engineering a cell to be resistant to an oligonucleotide comprising introducing a mutation in the region of the genome of the cell targeted by the oligonucleotide.

2. The method of claim 1, wherein the mutation is introduced using a genome editing method.

3. The method of claim 2, wherein the genome editing method is selected from the group consisting of CRISPR/Cas system, Cre/Lox system, TALEN system, ZFNs system and homologous recombination.

4. The method of claim 3, wherein the CRISPR-mediated genome editing comprises introducing into the cell a first nucleic acid encoding a Cas9 nuclease, a second nucleic acid comprising a guide RNA (gRNA), wherein the gRNA is specific to the region of the genome of the cell targeted by the oligonucleotide, and a third nucleic acid that will act as a template for homologous recombination, wherein the third nucleotide comprises the mutation.

5. The method of any one of the preceding claims, wherein the oligonucleotide targets a coding region of a gene, and the mutation in the genome of the cell comprises a silent mutation in the targeted coding region of the gene.

6. The method of any one of the preceding claims, wherein the oligonucleotide targets a noncoding region of the genome, and the mutation in the genome of the cell comprises a mutation in the targeted noncoding region of the gene.

7. The method of any one of the preceding claims, wherein the mutation comprises a deletion in the region of the genome of the cell.

8. The method of any one of the preceding claims, wherein the at least one oligonucleotide targets an essential gene that is needed for survival of a cell, thereby killing the cell.

9. The method of any one of the preceding claims, wherein the at least one oligonucleotide is selected from an antisense oligonucleotide, a small interfering RNA (siRNA), an RNAi, a microRNA, an artificial microRNA, and a ribozyme.

10. The method of any one of the preceding claims, wherein the at least one oligonucleotide is between 12 and 30 nucleotides in length.

11. The method of claim 10, wherein the at least one oligonucleotide is between 16 and 24 nucleotides in length.

12. A cell engineered to be resistant to an oligonucleotide comprising a mutation in the region of the genome of the cell targeted by the oligonucleotide.

13. The cell of claim 12, wherein the oligonucleotide targets a coding region of a gene, and the mutation in the genome of the cell comprises a silent mutation in the targeted coding region of the gene.

14. The cell of any one of claim 12 or 13, wherein the oligonucleotide targets a noncoding region of the genome, and the mutation in the genome of the cell comprises a mutation in the targeted noncoding region of the gene.

15. The cell of any one of claim 12 or 13, wherein the mutation comprises a deletion in the region of the genome of the cell targeted by the oligonucleotide.

16. The cell of any one of claim 12 or 13, wherein the at least one oligonucleotide is between 12 and 24 nucleotides in length.

17. The cell of any one of claim 12 or 13, wherein the at least one oligonucleotide is selected from an antisense oligonucleotide, a small interfering RNA (siRNA), an RNAi, a microRNA, an artificial microRNA, and a ribozyme.

18. The cell of any one of claim 12 or 13, wherein the cell is a mammalian cell.

19. The cell of any one of claim 12 or 13, wherein the cell is a human cell.

20. The cell of any one of claim 12 or 13, wherein the cell is a mesenchymal stem cell.

21. A method of delivering at least one oligonucleotide to a subject comprising administering to the subject a carrier cell loaded with the at least one oligonucleotide, wherein the carrier cell is engineered to be resistant to the at least one oligonucleotide.

22. The method of claim 21, wherein the at least one oligonucleotide is designed to inhibit the growth of a cell or to kill a cell.

23. The method of claim 21, wherein the carrier cell forms a gap junction with a target cell of the subject; thereby delivering the at least one oligonucleotide into the target cell through the gap junction.

24. The method of claim 21, wherein the carrier cell delivers the oligonucleotide to a target cell of the subject through an exosome containing the oligonucleotide, which is endocytosed into the target cell.

25. The method of any one of claims 21-24, wherein the carrier cell is loaded with the at least one oligonucleotide as a result of transfection with the at least one oligonucleotide.

26. The method of any one of claims 21-24, wherein the carrier cell is loaded with the at least one oligonucleotide as a result of expression of the at least one oligonucleotide from an exogenous nucleic acid encoding the oligonucleotide.

27. The method of any one of claims 21-26, wherein the at least one oligonucleotide targets an essential gene that is needed for survival of a cell, thereby killing the cell.

28. The method of any one of claims 21-27, wherein the at least one oligonucleotide targets a gene that is specifically needed for the survival of the target cell, thereby killing only the target cell.

29. The method of any one of claims 21-28, wherein the at least one oligonucleotide is between 12 and 24 nucleotides in length.

30. The method of any one of claims 21-29, wherein the at least one oligonucleotide is selected from an antisense oligonucleotide, a small interfering RNA (siRNA), an RNAi, a microRNA, an artificial microRNA, and a ribozyme.

31. The method of claim 27, wherein the carrier cell is engineered to comprise a silent mutation in the essential gene such that the at least one oligonucleotide cannot target the essential gene of the carrier cell.

32. The method of claim 31, wherein the carrier cell comprises a deletion in the essential gene such that the at least one oligonucleotide cannot target the essential gene of the carrier cell.

33. The method of any one of claims 21-31, wherein the carrier cell and the target cell each expresses connexin 43, connexin 40 or a member of an alpha connexin family to form a gap junction.

34. The method of any one of claims 21-33, wherein the carrier cell comprises a mutation in the region of the genome of the cell targeted by the oligonucleotide introduced using a genome editing method.

35. The method of claim 34, wherein the genome editing method is selected from the group consisting of CRISPR/Cas system, Cre/Lox system, TALEN system, ZFNs system and homologous recombination.

36. The method of claim 35, wherein the CRISPR-mediated genome editing comprises introducing into the cell a first nucleic acid encoding a Cas9 nuclease, a second nucleic acid comprising a guide RNA (gRNA), wherein the gRNA is specific to the region of the genome of the cell targeted by the oligonucleotide, and a third nucleic acid that will act as a template for homologous recombination, wherein the third nucleotide comprises the mutation.

37. The method of any one of claims 23-24, wherein the target cell is a cancer cell.

38. The method of claim 37, wherein the cancer cell is part of a solid tumor.

39. The method of any one of claims 23-24, wherein the target cell is syncytial to at least one other target cell.

40. The method of any one of claims 21-39, wherein the carrier cell is a mammalian cell.

41. The method of any one of claims 21-39, wherein the carrier cell is a mesenchymal stem cell.

42. The method of any one of claims 21-39, wherein the carrier cell and the target cells are human cells.

43. A method of treating a subject suffering from cancer comprising administering to the subject a carrier cell loaded with at least one oligonucleotide designed to kill a cancer cell, wherein the carrier cell is resistant to the killing effects of the at least one oligonucleotide.

44. The method of claim 43, wherein the carrier cell is loaded with the at least one oligonucleotide as a result of transfection with the at least one oligonucleotide.

45. The method of claim 43, wherein the carrier cell is loaded with the at least one oligonucleotide as a result of expression of the at least one oligonucleotide from an exogenous nucleic acid encoding the oligonucleotide.

46. The method of any one of claims 43-45, wherein the at least one oligonucleotide targets an essential gene that is needed for survival of a cell, thereby killing the cell.

47. The method of any one of claims 43-45, wherein the at least one oligonucleotide targets a gene that is specifically needed for the survival of a cancer cell, thereby killing only the cancer cell.

48. The method of any one of claims 43-47, wherein the at least one oligonucleotide is between 12 and 24 nucleotides in length.

49. The method of any one of claims 43-48, wherein the at least one oligonucleotide is selected from an antisense oligonucleotide, a small interfering RNA (siRNA), an RNAi, a microRNA, an artificial microRNA, and a ribozyme.

50. The method of any one of claims 43-49, wherein the carrier cell comprises a silent mutation in the gene that the at least one oligonucleotide targets such that the at least one oligonucleotide cannot target the gene of the carrier cell.

51. The method of any one of claims 43-50, wherein the carrier cell comprises a deletion in the essential gene such that the at least one oligonucleotide cannot target the essential gene of the carrier cell.

52. The method of any one of claims 43-51, wherein the carrier cell and the cancer cell each expresses connexin 43, connexin 40 or a member of an alpha connexin family to form the gap junction.

53. The method of any one of claims 43-52, wherein the cancer cell is syncytial to at least one other target cell.

54. The method of any one of claims 43-53, wherein the carrier cell is a mammalian cell.

55. The method of claim 54, wherein the subject is a human.

56. The method of claim 55, wherein the carrier cell is a human mesenchymal stem cell.
